(19) United States
(12) Patent Application Publication
(10) Pub. No.: US 2023/0337943 A1
(43) Pub. Date: Oct. 26, 2023

(54) PEG SENSING APPARATUS AND METHODS OF USE

(71) Applicant: Qr8 Health Inc., Cleveland, OH (US)

(72) Inventors: Christopher Pekrul, Boston, MA (US); Kenneth Rhodes, Boston, MA (US); Sean Robertson, Boston, MA (US); David Schindler, Cleveland, OH (US)

(21) Appl. No.: 18/333,433

(22) Filed: Jun. 12, 2023

Related U.S. Application Data
(63) Continuation-in-part of application No. 18/065,278, filed on Dec. 13, 2022.
(60) Provisional application No. 63/289,844, filed on Dec. 15, 2021.

(51) Int. Cl. A61B 5/11; A61B 5/00

(52) U.S. Cl. CPC A61B 5/1124; A61B 5/4082; A61B 5/6887; A61B 5/1101; A61B 5/112

(57) ABSTRACT

A movement-assessment device and methods for using the testing device includes a peg board device having a plurality of apertures on a top surface, and a plurality of photo-optical gate sensors. A computing device, comprising a touchscreen interface, communicates with the peg board device.

### CROSS-REFERENCE TO RELATED APPLICATIONS

This application is a continuation-in-part of U.S. patent application Ser. No. 18/065,278, filed on Dec. 13, 2022, which claims priority to U.S. Provisional Patent Application No. 63/289,844, filed on Dec. 15, 2021.

### BACKGROUND

Movement disorders may be described as a broad set of neurological diseases or conditions characterized primarily by abnormal movement of an affected individual. Such movement may either manifest via tremor or be slower, faster, or less smooth than the movement of a healthy individual. The assessment of movement disorders is traditionally done with subjective tests through numerous pieces of analog equipment. Increased adoption of handheld, wearable, and mobile technology provides an opportunity to streamline traditional assessments, provide more robust detail and data, and provide new metrics that are beneficial and desirable for clinicians and researchers in the treatment of neurological disorders.

The Nine Hole Peg Test is an assessment used to measure finger dexterity in individuals with various types of neurological diseases or conditions. It is a rectangular board, typically made of plastic or wood, with nine holes laid out in a grid pattern placed 32 mm apart on the top surface and a dish at one end of the board for holding pegs. An individual performs the test by removing the pegs from the dish, one at a time, and placing each one in a hole on the peg board; once pegs have been placed in all of the holes, the individual proceeds to remove the pegs from each hole, one at a time, and place them back in the dish. This is done with one hand at a time as quickly as possible. The primary outcome is time to complete the task.

Recently, this apparatus has been modified to be compatible with a commercially available tablet, such as an iPad, by laying a peg board on top of the tablet. The test is performed in the manner outlined above, with time to complete being the primary outcome. Digitizing the task, however, provides greater quality and quantity of data. For example, the tablet may easily detect the removal and insertion of each peg, store this information on the device, and subsequently transmit the data to a cloud-based database. This approach improves the test by providing clinicians and researchers with additional types of data and a more accurate measurement of performance. Despite this improvement, this design maintains certain limitations, most notably being the software and physical dimensions of the accompanying tablet. Consumers of the tablet-based peg board must be aware of changing software functionality and tablet size, which may be difficult to anticipate and result in costly changes to manufacturing specifications and processes. Therefore, it is desirable to provide a device that may provide at least the same amount of data collection and quality in a manner that is independent of tablet size or operating system.

### BRIEF DESCRIPTION OF THE DRAWINGS

FIG. 1 is a top view of a peg board device during a manual dexterity task, according to some embodiments of the present disclosure.

FIG. 2 is a side perspective view of a peg board device during a manual dexterity task, according to some embodiments of the present disclosure.

FIG. 3 is a top view of the peg board device with the storage dish cover opened to exposes the pegs and remote in the storage dish, according to some embodiments of the present disclosure.

FIG. 4 shows a peg board device, remote, and external mobile device arranged for a patient to perform a manual dexterity task, according to some embodiments of the present disclosure.

FIG. 5 shows a patient performing a manual dexterity task with a peg board device, according to some embodiments of the present disclosure.

### SUMMARY

The present disclosure relates to a remote peg-sensing device configured to assess a movement disorder through various sensors, which may include sensors configured to detect insertion of pegs into a plurality of holes or apertures on one surface of the peg-sensing device. The present disclosure also relates to a peg-sensing device that may transmit data to an information storage device such as a computer, tablet, or other electronic apparatus.

### DETAILED DESCRIPTION

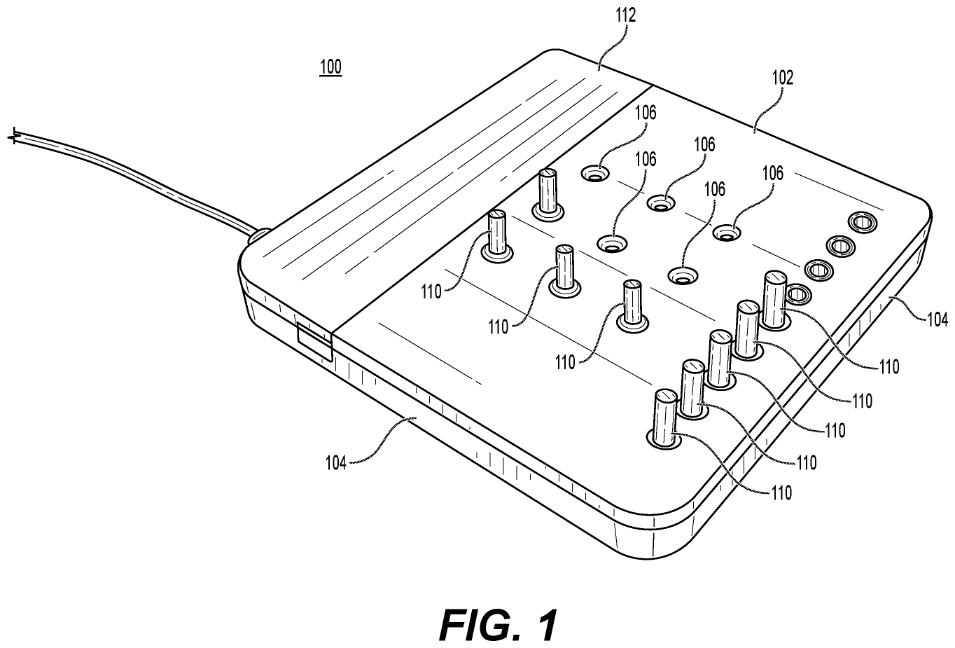

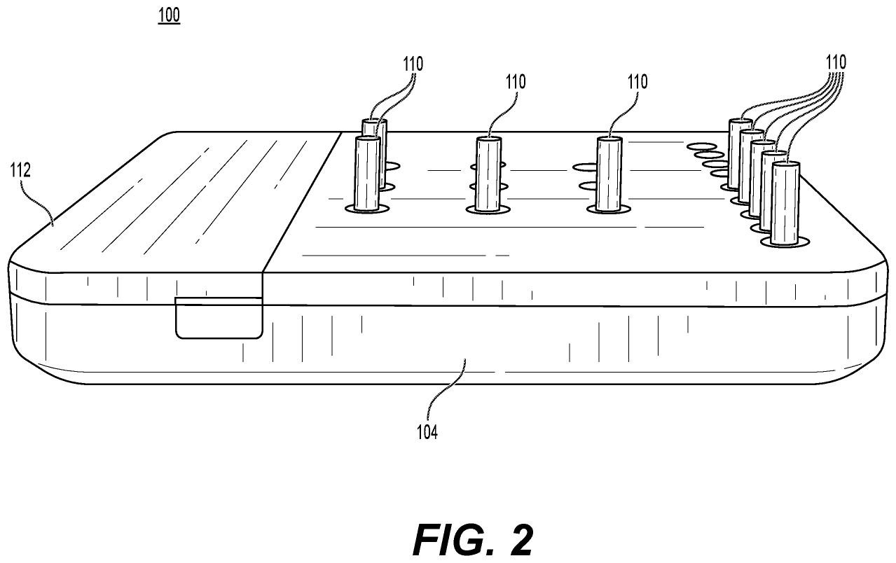

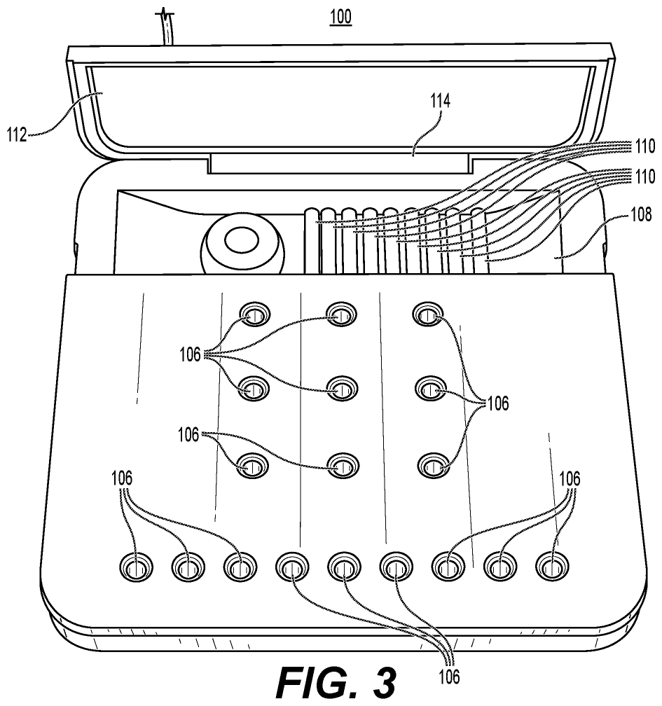

The present disclosure relates generally to various embodiments of a peg board device. FIGS. 1-3 are multiple views of a peg board device during a manual dexterity task, according to some embodiments of the present disclosure. As shown in FIGS. 1-3, the peg board device 100 may include a top surface 102, a bottom surface (not shown), and side surfaces (104). The top surface 102 may include a plurality of holes or apertures 106.

Additionally, the top surface may have a dish 108 for storing a plurality of pegs 110, each peg 110 sized to fit within a hole or an aperture 106 on the top surface 102 of the peg board device 100, and a cover 112 for the dish 108 for secure storage. The cover 112 may attach to the peg board device 100 by any suitable mechanism, such as one or more magnets or a hinge 114. The pegs 110 may include one or more internal sensors, such as a gyroscope, accelerometer, magnetometer, or a combination thereof. The internal sensors may be used determine the relative and absolute motion of the pegs 110. The pegs 110 may further comprise at least one processor, and may be capable of transmitting the data captured with the internal sensors wirelessly via protocols, such as Zigbee, Bluetooth, or Bluetooth Low Energy (BLE).

The types of metal which may be used for the peg board device 100 includes one or more of aluminum, steel, stainless steel, copper, zinc, magnesium, or other alloys of the aforementioned metals. The types of plastics that may be used for the peg board device includes polyethylene terephthalate, high-density polyethylene, polyvinyl chloride, low-density polyethylene, polypropylene, polystyrene, polycarbonate, polyketide, acrylic, acrylonitrile, butadiene, styrene, fiberglass, nylon, or a combination thereof. The types of metal which may be used for the pegs includes one or more of aluminum, steel, stainless steel, copper, zinc, magnesium, or other alloys of the aforementioned metals. The types of plastics that may be used for the pegs includes polyethylene terephthalate, high-density polyethylene, polyvinyl chloride, low-density polyethylene, polypropylene, polystyrene, polycarbonate, polyketide, acrylic, acrylonitrile, butadiene, styrene, fiberglass, nylon, or a combination thereof.

The peg board device 100 may comprise one or more movement sensors, such as photo-optical gate sensors. The photo-optical gates may be configured to sense a peg 110 being inserted or removed from one of the plurality of holes 106 on the top surface 102. In some embodiments, the photo-optical gates may be an Omron EE-SX1070 photomicrosensor, or a photomicrosensor with similar technical specification.

In some embodiments, the determination of a peg 110 being inserted or removed from one of the plurality of holes 106 may be achieved by interaction via Bluetooth (e.g., Apple's “Made for iPhone/iPod/iPad” program, or “MFI”). In some embodiments, the pegs 110 may be manufactured so that it can trick the touchsreen into thinking that the peg 110 is being touched by a hand. This may be achieved by creating a capacitive coupling between the peg 110 and the touchscreen. In some embodiments, the peg 110 may create a capacitive coupling between the peg 110 and the screen.

Additionally, the peg board device 100 may comprise an embedded circuit device to process and record data gathered by the photo-optical gate sensors, and may transmit such data to a computing device, such as a mobile tablet, handheld device, or desktop computer. In some embodiments, a printed circuit board (PCB) device may connect the sensors to the embedded circuit. The peg board 100 may interact with a computing device, such as a mobile device, handheld device, laptop computer, or desktop computer via wireless communication, such as Bluetooth low energy (BLE).

The peg board device 100 may comprise a rechargeable battery. The peg board device 100 may further comprise a battery capable of charging wirelessly. In some embodiments, the peg board device 100 may charge external peripheral device by means of wireless charging.

In some embodiments, a patient may perform a manual dexterity task with the peg board device 100 by removing each of nine pegs 110, one at a time, from a dish 108 on the top surface 102 of the peg board device 100, and inserting each peg 110 into one of nine holes 106 laid out in a grid pattern at even intervals. Once all of the pegs 110 have been inserted into the grid, the patient begins removing the pegs 110 from the grid, one at a time, and placing them back into the dish 108. In some embodiments, the dish 108 may be positioned at different locations or take forms other than a dish, for example, on either side of the grid patterns, or on one of the side surfaces 104 in the form of a drawer.

In some embodiments, a patient may perform a manual dexterity task with the peg board device 100 by removing each of nine pegs 110, one at a time, from a linear row of holes 106 on the top surface 102 of the peg board device 100, and inserting each peg 110 into one of nine holes 106 laid out in a grid pattern at even intervals. Once all of the pegs 110 have been inserted into the grid, the patient begins removing the pegs 110 from the grid, one at a time, and placing them back into the linear row.

The peg board device 100 may gather information and data from tasks, such as the stated embodiments to be stored, processed, analyzed, or further transferred to an external device, such as a computing device. Such transfer of data may be either by means of wired or wireless communication.

The peg board device 100 may additionally interact with a plurality of peripheral devices to augment data capture capabilities and the type of tasks that may be completed. Such additional devices may acquire and/or assess other parameters of movement disorders or neurological disorders.

By way of example, a device to assess a patient's balance while performing a manual dexterity task via the peg board device 100 may interact with the peg board device 100. Examples of such a balance device include a mobile computing device or other wearable devices, such as a smart watch, activity tracker, or other inertial sensor device. In some embodiments, a balance device is a force plate, force plate treadmill, or other dynamic multi-sensory system for assessing kinematics.

In some embodiments, the combined results of such a dual task or a multi task may then be used to treat, diagnose, alter treatment, and/or manage a patient with a movement disorder, neurological disorder, or cognitive disorder. In one example test paradigm, a patient may be performing a balance task on a dynamic multi-sensory system while simultaneously performing a manual dexterity task with the peg board device. The patient's performance at such a dual task may be compared to standardized scores, or used to track a patient's progress over time. Such conditions include movement disorders (including but not limited to Parkinson's, Essential Tremor, Dystonia, Tourette's and Progressive Supranuclear Palsy), autism, heart failure, heart disease, traumatic brain injury, stroke, vestibular disease, migraines, dementia, amyotrophic lateral sclerosis (ALS), and attention deficit disorder (ADD).

Furthermore, embodiments of the present disclosure include apparatuses and methods for the assessment of movement as it relates to treatment with deep-brain stimulation (DBS) therapies, assessment of fall risk, assessment of frailty, assessment of pharmaceuticals and the assessment of various psychological disorders.

Furthermore, embodiments of apparatuses and methods for performing tasks assessing action tremor, resting tremor, postural tremor, gait, balance, or a tapping test are included herein.

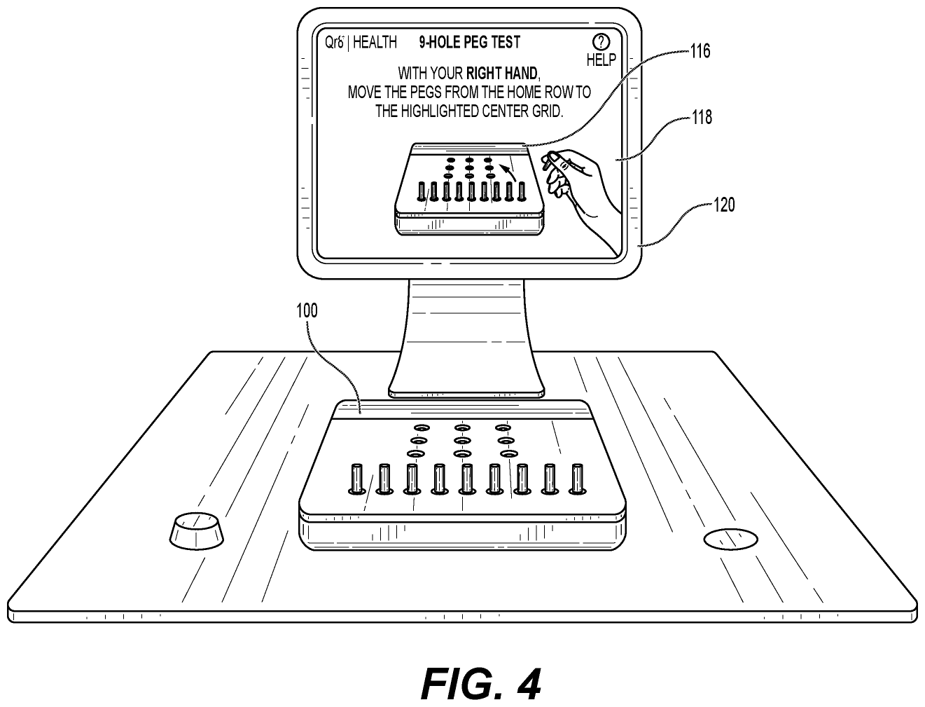

FIG. 4 shows a peg board device, remote, and external mobile device arranged for a patient to perform a manual dexterity task, according to some embodiments of the present disclosure. As shown in FIG. 4, the peg board device 100 may interact with a mobile application (“mobile app”) or other computer program. In some embodiments, the mobile app provides instructions for a manual dexterity task to be completed by a patient. In some embodiments, the mobile app has a graphical representation 116 of the pegs 110 being inserted and removed during a manual dexterity task. In some embodiments, the peg board device 100 interacts with a mobile device, via a wireless communication protocol, equipped with a camera to record a patient performing or completing a manual dexterity task with the peg board device 100. The peg board device 100 may detect test initiation by the insertion of a first peg, the initial movement of a first peg, a patient tapping on a touch senor or button, or a combination thereof. The patient may also initiate the test by tapping on a touchscreen device 118 or using a remote.

In some embodiments, a camera and one or more processors are used to provide augmented reality (AR) or virtual reality (VR) and image processing for the tracking of a patient's movement or the movement of the pegs 110 for the completion of a corresponding manual dexterity task. In some embodiments, the AR or VR is used in combination with the peg board device 100 and one or more additional peripheral devices to complete a dual task or a multi task. Examples of such additional peripheral devices include a force plate, force plate treadmill, or other dynamic multi-sensory kinematic system. In certain embodiments, a patient may perform a balance task with a dynamic multi-sensory kinematic system, an AR or VR capable headset, and a manual dexterity task with the peg board device 100.

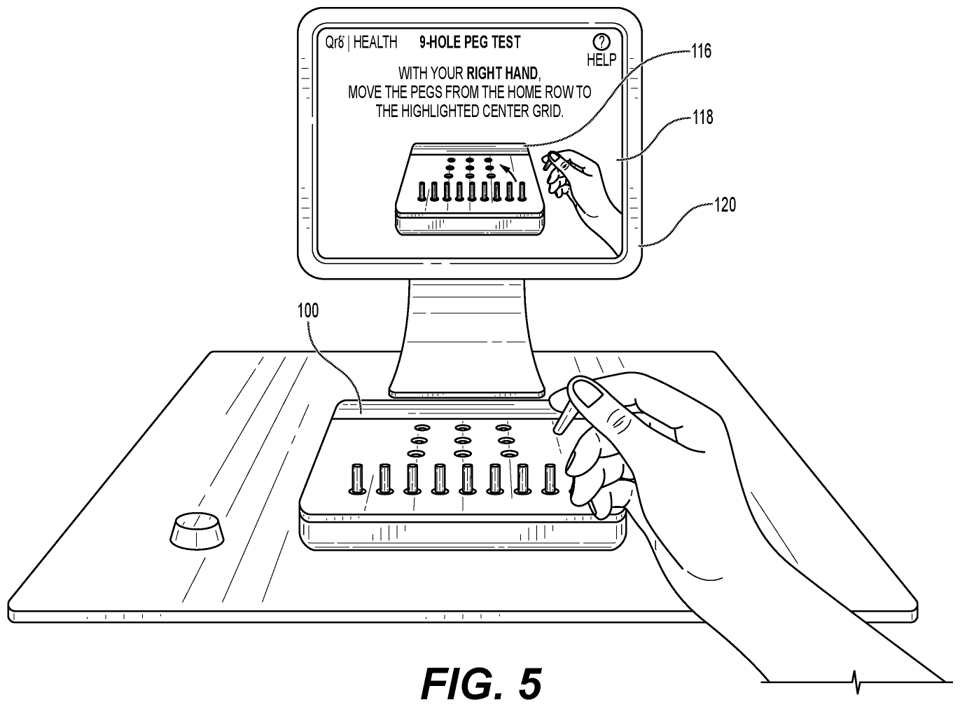

FIG. 5 shows a patient performing a manual dexterity task with a peg board device, according to some embodiments of the present disclosure. The present disclosure is also related to a system for assessing the symptoms of a movement disorder or side effects of an intervention of movement disorders or cognitive disorders in a patient. As shown in FIG. 5, the system may include a computer or a mobile device 120, such as a tablet, configured to have multiple mobile applications on it and a peg board device 100. The system may measure a plurality of symptoms that may be in different domains, such as motor symptoms, cognitive symptoms, or mood that may be caused by the disease or an intervention, such as pharmaceuticals or devices including devices for Deep Brain Stimulation.

Embodiments of the present disclosure may also assess patients with other conditions that would require a plurality of assessments in different domains of symptoms. The conditions may include but not limited to autism, heart failure, heart disease, stroke, traumatic brain injury, vestibular disease, migraines, dementia, ALS and attention deficit disorder (ADD).

Interventions for movement disorders may have positive effects on symptoms, such as tremor, and may cause side effects, such as cognitive issues. For example, titration of pharmaceuticals or stimulation parameters during deep brain stimulation may be difficult given all the different parameters and domains that need to be assessed as well as the number of potential therapeutic options, such as stimulation settings that are available.

In some embodiments, a method of titrating treatment for a patient with a movement disorder using the peg board device 100 consistent with the present disclosure is completed by the patient to assess a patient's movement or motor function. In some embodiments, based on this assessment, both an Effect Score and a Side Effect Score are calculated by via an algorithm on the mobile device 120, the peg board device 100, or a combination thereof. A treatment may be given to the patient based on the assessment, and the treatment may include a pharmaceutical or stimulation from a stimulation device. For example, a dose of the treatment may be entered into an iPad.

In some embodiments, a device may suggest an alternative treatment plan for the patient. In some embodiments, the patient may be given a different treatment, such as a different set of stimulation parameters or a different dose of a treatment.

In some embodiments, after an appropriate washout period to allow for the old treatment stop and the new treatment to take place, a second assessment or additional assessments may be performed again by the patient. Based on the second assessment, both the effect score and side effect score may be calculated. In some embodiments, the first set of scores may be compared with the second set of scores in order to make a treatment decision about the dose or stimulation. In some embodiments, the system may suggest one of: the original parameters, the second parameters, or new parameters to test. This process may be completed a plurality of times over a duration of time. The duration of time may include time intervals and durations of a day, a week, a month, several months, or one or more years to titrate the patient's treatment.

In some embodiments, given the fact that many neurologic diseases are degenerative, assessments may need to be performed periodically throughout the life of the patient. In some embodiments, the starting point may be with an initial treatment. In some embodiments, the initial treatment may be assessed by comparing a patient's score on a first task or set of tasks with that patient's score on a second task or set of tasks. In some embodiments, the starting point may be with no treatment.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term

does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

What is claimed is:

1. A system for assessing movement, the system comprising:
a peg board device comprising a plurality of apertures on a top surface, a plurality of photo-optical gate sensors within the plurality of apertures, a printed circuit board, and an embedded circuit device connected to the photo-optical gate sensors via the printed circuit board; and
a computing device, comprising a touchscreen interface, a non-transitory computer-readable medium configured to store instructions; and a processor configured to execute instructions.

2. The system of claim 1, wherein the top surface comprises a recessed area.

3. The system of claim 2, wherein the recessed area is configured to receive one or more pegs.

4. The system of claim 1, wherein the peg board device is configured for a patient to perform a manual dexterity task.

5. The system of claim 1, wherein the manual dexterity task is a nine-hole peg test.

6. The system of claim 1, wherein the peg board device further comprises a plurality of pegs.

7. The system of claim 6, wherein one of more of the plurality of pegs is provided with an accelerometer, a gyroscope, a magnetometer, or combination thereof.

8. A method of treating a patient for a movement or cognitive disorder, the method comprising:
administering a manual dexterity task with a peg board device to a patient, the peg board device comprising:
a plurality of apertures on a top surface, a plurality of pegs, a plurality of photo-optical gate sensors within the plurality of apertures, a printed circuit board, and an embedded circuit device connected to the photo-optical gate sensors via the printed circuit board; and
a computing device comprising a touchscreen interface, a non-transitory computer-readable medium to store instructions, and a processor configured to execute instructions; and
instructing the patient to insert the plurality of pegs one at a time into the plurality of apertures, and to remove such pegs one at a time from the plurality of apertures.

9. The method of claim 8, wherein the computing device is configured to provide video instructions for the patient to perform the manual dexterity task with the peg board device.

10. The method of claim 8, further comprising instructing the patient to performs a dual task including the manual dexterity task.

11. The method of claim 8, wherein the dual task is a balance task.

12. A device comprising:
a top surface, a bottom surface, and a plurality of sides, the top surface comprising a plurality of apertures and a recessed area;
a plurality of pegs; and
a rechargeable battery.

13. The device of claim 12, wherein one or more of the plurality of pegs is provided with an accelerometer, a gyroscope, a magnetometer, or combination thereof.
